(19) United States
(12) Patent Application Publication
(10) Pub. No.: US 2005/0106263 A1
(43) Pub. Date: May 19, 2005

(54) Green tea, multivitamin, mineral and herb based hair and male facial skin formulas

(76) Inventor: Yucel Donmez, Chicago, IL (US)

Correspondence Address: YUCEL DONMEZ, 3RD FLOOR, 5520 N. ARTESIAN AVE, CHICAGO, IL, 60625, US

(21) Appl. No.: 10/717,002

(22) Filed: Nov. 19, 2003

(51) Int. Cl. A61K 35/78; A61K 31/7048; A61K 31/4188; A61K 31/555; A61K 31/525; A61K 31/51; A61K 33/32

(52) U.S. Cl. 424/643; 424/729; 514/184; 514/251; 514/27; 514/393; 514/356; 514/276; 514/561

(57) ABSTRACT

The present invention is a natural formulation for treatment of male, female and adolescent pattern hair loss. The formulation contains a combination of Green Tea leaf extract, Polyphenols, Epigallocatechin Gallate (EGCG), Vitamin E, Folic Acid, Copper (as Amino Acid Chelate), B12, Zinc (as Oxide), Calcium Pantothenate, Niacin, Biotin, Riboflavin, Thiamine, and optionally Inositol, Black Tea Extract and Nettle Extract. The various extracts are prepared according to traditional procedures, and then combined in a suitable formulation for administration to the patient for treatment of male, female and adolescent pattern hair loss.

### REFERENCES CITED

U.S. Patent Documents

| | | | |
|---|---|---|---|
| 5,972,345 | Oct. 26, 1999 | Chizick, et al. | 424/727 |
| 6,299,925 | Oct. 9, 2001 | Xiong, et al. | 426/597 |
| 6,455,057 | Sep. 24, 2002 | Barrett, et al. | 424/401 |
| 6,358,541 | Mar. 19, 2002 | Goodman | 424/727 |

### OTHER REFERENCES

1—J Leukoc Biol. 2001 May;69 (5):719-26.

Green tea polyphenol (−)-epigallocatechin-3-gallate treatment to mouse skin prevents UVB-induced infiltration of leukocytes, depletion of antigen-presenting cells, and oxidative stress.

Katiyar S K, Mukhtar H.Department of Dermatology, School of Medicine, Case Western Reserve University, Cleveland, Ohio 44106, USA

2—Carcinogenesis. 2001 Feb;22 (2):287-94

Green tea polyphenol (−)-epigallocatechin-3-gallate treatment of human skin inhibits ultraviolet radiation-induced oxidative stress.

Katiyar S K, Afaq F, Perez A, Mukhtar H.Department of Dermatology, Volker Hall 501, 1530 3rd Ave S, The University of Alabama at Birmingham, Birmingham, Ala. 35294-0019, USA.

3—Carcinogenesis. 1999 Nov; 20 (11):2117-24.

Prevention of UVB-induced immunosuppression in mice by the green tea polyphenol (−)-epigallocatechin-3-gallate may be associated with alterations in IL-10 and IL-12 production.

Katiyar S K, Challa A, McCormick T S, Cooper K D, Mukhtar H.

Department of Dermatology, Case Western Reserve University, 11100 Euclid Avenue, Cleveland and University Hospitals of Cleveland and Va. Hospital, Cleveland, Ohio 44106, USA. sxk32@po.cwru.edu

4—Int J Oncol. 2002 Dec; 21 (6):1213-22

Treatment of silymarin, a plant flavonoid, prevents ultraviolet light-induced immune suppression and oxidative stress in mouse skin.

Katiyar S K. Department of Dermatology, University of Alabama at Birmingham, Birmingham, Ala. 35294-0019, USA. skatiyar@uab.edu

5—Photochem Photobiol. 1999 Feb; 69 (2):148-53.

Polyphenolic antioxidant (−)-epigallocatechin-3-gallate from green tea reduces UVB-induced inflammatory responses and infiltration of leukocytes in human skin.

Katiyar S K, Matsui M S, Elmets C A, Mukhtar H.

Department of Dermatology, Case Western Reserve University, Cleveland, Ohio 44106, USA.

6—Photochem Photobiol. 1999 Feb; 69 (2):148-53.

Green tea polyphenols: DNA photodamage and photo immunology.

Katiyar S K, Bergamo B M, Vyalil P K, Elmets C A.

Department of Dermatology, School of Medicine, University of Alabama at Birmingham, 1670 University Blvd., VH501, Box 202, Birmingham, Ala.35294

7—Carcinogenesis. 1998 Dec; 19 (12):2201-4.

(−)-Epigallocatechin-3-gallate inhibition of ultraviolet B-induced AP-1 activity.

Barthelman M, Bair W B 3rd, Stickland K K, Chen W, Timmermann B N, Valcic S, Dong Z, Bowden G T. Department of Radiation Oncology, University of Arizona Health Sciences Center, Tucson 85724, USA.

8—Carcinogenesis. 2003 May; 24 (5):927-36

Treatment of green tea polyphenols in hydrophilic cream prevents UVB-induced oxidation of lipids and proteins, depletion of antioxidant enzymes and phosphorylation of MAPK proteins in SKH-1 hairless mouse skin.

Vayalil P K, Elmets C A, Katiyar S K.

Department of Dermatology, University of Alabama at Birmingham, 1670 University Blvd, Volker Hall 557, 35294, USA.

9—Free Radic Biol Med. 2002 Oct 15; 33 (8):1097-105.

Green tea polyphenol epigallocatechin-3-gallate inhibits the IL-1 beta-induced activity and expression of cyclooxygenase-2 and nitric oxide synthase-2 in human chondrocytes.

Ahmed S, Rahman A, Hasnain A, Lalonde M, Goldberg V M, Haqqi T M.

### FIELD OF THE INVENTION

The present invention per claims 1 & 2 is directed to a preparation for treatment of male, female and adolescent pattern hair loss, and in particular, to a natural herbal, vitamin and mineral preparation to help stop further hair loss and increase hair growth by achieving and maintaining healthy scalp condition.

The present invention per claim 3 is directed to a preparation for treatment of male facial skin conditions by assisting in the prevention of male facial skin damage due to UV, cigarette smoke and other environmental effects, wrinkles and discoloration. This formulation also assists in the reversal of the aforementioned damage.

### BACKGROUND OF THE INVENTION

Hair Loss

Research has been done on hair loss for 5500 years. Hundreds of drugs and supplements are currently on the market, but few are successful. Most men and women experience hair loss as they get older; indeed, most men have some degree of baldness by age 60. This is quite normal and affects some persons more than others, especially if baldness runs in the family. Sudden or abnormal hair loss could, however, result from:
Taking certain medications (like some used in treating cancer, circulatory disorders, ulcers or arthritis)
Following a crash diet
Hormonal changes such as with menopause
A prolonged or serious illness some medical conditions lead to hair loss such as Hypothyroidism and Ringworm (the latter is a fungal infection that effects the scalp and/or the hairs themselves). These require medical treatment.
Areata, which causes areas of patchy hair loss, but does not affect the scalp. This condition improves rapidly when treated, and can even disappear within 18 months without treatment. Doctors may prescribe a topical steroid to be used once or twice a day. For cosmetic reasons, some older persons wear wigs or toupees. Surgical hair transplant operations and the medication Rogaine are treatment options for both men and women, in very select cases.

Human hair undergoes a normal growth cycle where each hair grows continuously for approximately 2 to 4 years, stops growing for 2 to 4 months then falls out. In its place a new healthy hair begins to grow and this cycle is repeated. The hairs on the head are always in different stages of the cycle, so it is normal to lose hair everyday. On average, up to about 100 hairs is lost per day.

In male pattern hair loss, the normal hair growth cycle is disrupted and more than the average number of hairs are shed per day without having the old hairs replaced by new ones. Male pattern hair loss is determined by a combination of male hormones (androgens) and heredity. Men susceptible to male pattern baldness usually experience the onset sometime in their 20's and it becomes more common as they age. Androgenetic alopecia is the most common type of hair loss in men, with approximately 50% of men experiencing this hair loss to some degree by the age of 50.

In addition to adrogenetic alopecia, other factors may influence hair loss, many of which are temporary. Amongst these factors include stress of an illness or major surgery, medicines, such as those used in chemotherapy, blood thinners, antidepressants, excessive amounts of vitamin A and certain disease states like diabetes.

There is increasing evidence of the link between male pattern hair loss and the level of 5 alpha-reductase. 5 alpha-reductase converts the hormone testosterone into dihydrotestosterone (DHT).

There have been many reports of men with male pattern hair loss having increased levels of DHT in the scalp. It appears that DHT contributes to the shortening of the growth phase and thinning of the hair.

No potion or ointment exists that will produce a full head of hair. The only remedy that comes close is the medication Rogaine, originally developed as a blood pressure medication. Rogaine has shown promising results for some (but not all) cases of baldness. This applies to men and women. However although many Rogaine users claim hair rejuvenation, when they have stopped using Rogaine most if not all of their new hair reverted to the original state.

Research Finds Preventive Qualities, Damage Reversal, in Green Tea

Green tea extract is an amazingly powerful antioxidant, which makes it a benefit to both immune and circulatory health. Used primarily for its free radical fighting capabilities, Green Tea extract has many other beneficial properties that are just beginning to be discovered.
EGCG protects against digestive and respiratory infections
Potent groups of antioxidants from the catechin family which: scavenge free radicals and reduce the risk of many types of cancers; prevent DNA strand breaks; inhibit cell proliferation, decreasing the contact of carcinogens with cells; block cancer initiation, and slow cancer progression; help block the cancer-promoting actions of carcinogens, ultraviolet light, and metastasis
Reduces LDL-cholesterol levels thereby protecting the heart through prevention of vascular blood clotting
Reduces blood pressure (suppresses angiotensin I converting enzyme)
Reduces platelet aggregation
Inhibits pathogenic bacteria a primary cause of food poisoning
Blocks the attachment of the bacteria associated with dental cavities
Possesses antimicrobial properties that support immune-system health.

Drinking green tea may do more than just thwart a head cold, according to research presented Oct. 28, 2003 at the American Association for Cancer Research Second Annual International Conference on Frontiers in Cancer Prevention Research. Green tea already is believed to help lower cholesterol and prevent heart disease, fight bacteria and dental cavities, and possibly aid weight loss. New studies are now suggesting the various potential anti-cancer benefits of the age-old beverage. Research is now showing how this novel chemopreventive agent might work at the molecular level and in the human population

EGCG (epigallocatchin-3-gallate) is the most abundant and active chemopreventive agent in green tea, and has been associated with reduced risk of breast, pancreatic, colon, esophageal and lung cancers in humans. However, EGCG has a low oral bioavailability, meaning that to sustain effective levels for biological activity, individuals would need to drink at least seven to eight cups of tea a day, or ingest large amounts of green tea polyphenol extract. Researchers from SRI International in Menlo Park, Calif., have successfully synthesized several EGCG analogs that inhibit the in vitro (in an artificial environment) growth of tumor cell lines with potencies equal to or greater than EGCG itself.

The investigators developed a chemical synthesis of cis racemic EGCG that allows them to modify the A-, B- and D-rings of EGCG independently of each other. Using this method, they created several different analogs (a chemical compound structurally similar, but different in composition). SR 13196, an analog modified in the B- and D-rings, displays significantly more potent growth inhibition of breast cancer cell lines when compared to EGCG. SR 13193, an analog modified in the D-ring, inhibits expression of the potent angiogenic factor VEGF (vascular endothelial growth factor) in breast cancer cells, similar to EGCG.

“These analogs are not only valuable tools to clarify how green tea may fight cancer, but are also potential chemopreventive drug candidates themselves, with perhaps better pharmacokinetic properties than have been seen with EGCG thus far,” said SRI's Nurulain Zaveri, Ph.D., lead author of the study.

Researchers are aware that DNA is susceptible to damage by reactive oxygen species; 8-OHdG (8-hydroxydeoxyguanosine) is one of the most abundant lesions formed during this damage. The most established pathway to repair this type of lesion is via the human 8-oxoguanine glycosylase (hogg1), a base excision repair enzyme, of which there are several different forms. Researchers at the Arizona Cancer Center in Tucson conducted a phase lib randomized, controlled tea (green and black) intervention trial among heavy smokers, to study the effect of high consumption of tea (four cups per day) on oxidative DNA damage as measured by urinary 8-OHdG, and to evaluate the role of the hogg1 genotype as an effect modifier.

“We found no significant interaction between smoking, hogg1 genotypes, and tea intervention in terms of level of urinary 8-OHdG,” said Iman Hakim, M.D., Ph.D., of the Arizona Cancer Center and lead author of the study. “This suggests that green tea may be effective in decreasing levels of urinary 8-OHdG among smokers, regardless of their hogg1 genotype, thus reducing DNA damage that would potentially lead to tumor development,” she said.

Results demonstrate that green tea polyphenol treatment effectively inhibits phase I enzyme activities and enhances the phase II enzyme activities,” said Jia-Sheng Wang, M.D., Ph.D., of the Texas Tech University System, and lead investigator of the study.

The concept of prevention of cancer using naturally occurring substances that could be included in the diet consumed by the human population is gaining increasing attention. Tea, next to water, is the most popularly consumed beverage in the world and it is grown in about 30 countries. Abundant data, amassed from several laboratories around the world over the last ten years, provided convincing evidence that polyphenolic antioxidants present in tea afford protection against cancer risk in many animal-tumor bioassay systems. The epidemiological studies, though inconclusive, have also suggested that the consumption of tea is associated with a lowered risk of cancer. Much of this work has been done on green tea; less is known about black tea. Green tea contains many polyphenolic antioxidants, and Epigallocatechin Gallate (EGCG) is the key polyphenolic antioxidant believed to be responsible for most of the cancer chemopreventive properties of green tea.

Epigallocatechin Gallate (EGCG)

Epigallocatechin Gallate (EGCG) is a purified catechin separated from green tea. It is the main active component of the green tea polyphenol's biological activity. EGCG is a kind of catechin with the best anti-oxidation and free radical scavenging ability. By increasing cerebral cell metabolism it can also help to: prevent and postpone the occurrence of Alzheimer dementia; prevent and cure high blood lipid; prevent arteriosclerosis and cerebral thrombus; effect mid-term and later period cancers of the stomach and esophagus and prevent other cancers. It is the first remedy known to prevent and possibly cure cardiovascular and cerebrovascular diseases and gastric carcinoma. Many studies show EGCG and polyphenols inhibit accessory sex gland growth in laboratory animals. These results suggest that certain tea gallates can also regulate androgen action in target organs.

Vitamins

Vitamin E, Folic Acid, Copper (as Amino Acid Chelate), B12, Zinc (as Oxide), Calcium Pantothenate, Niacin, Biotin, Riboflavin and Thiamine all have been shown to have a therapeutic effect on hair and skin cells. Together with other anti-oxidants such as EGCG and polyphenols, this combination may increase the overall effectiveness all ingredients.

### SUMMARY OF THE INVENTION

The formulation claimed in this application includes as main ingredients: Green Tea Extract, Polyphenols, EGCG and other pertinent vitamins and minerals. It is believed that these ingredients improve the cells and tissues of both scalp and skin by healthy stimulation of the tissue and hair follicles. This in turn reduces hair loss and encourages new hair growth. The reduction of free radicals may also prevent cells from dying prematurely.

Over 200 peoples have tried this formula. More than 90% of these users claim hair loss ceased within one to two weeks. Of this group many subjects, both male and female, claimed new hair growth within 1 to 12 weeks. When subjects ceased to apply the formula for at least 4 months, they did not immediately start losing hair again. After hair loss stopped, subjects only needed to use formula once or twice a week to kept hair loss under control, while long-term users did not need to use the formula again for a considerable time. In comparison, Rogaine must be continually used or its effects will not only cease but will reverse. Rogaine also does not work well on the front hairline whereas the claimed formula does work on front hairline albeit at a slower rate than the top area of the scalp.

The present invention provides several variations of composition for treating male, female and adolescent pattern hair loss. One composition comprises Green Tea leaf extract, Polyphenols, Epigallocatechin Gallate (EGCG) Vitamin E, Folic Acid, Copper (as Amino Acid Chelate), B12, Zinc (as Oxide), Calcium Pantothenate, Niacin, Biotin, Riboflavin and Thiamine. Optionally, the composition may consist of other natural herb extracts such as nettle, black tea extract and Inositol.

### DETAILED DESCRIPTION

The present invention is a natural formulation for treatment of male, female and adolescent pattern hair loss. The formulation contains a combination of Green Tea leaf extract, Polyphenols, Epigallocatechin Gallate (EGCG), Vitamin E, Folic Acid, Copper (as Amino Acid Chelate), B12, Zinc (as Oxide), Calcium Pantothenate, Niacin, Biotin, Riboflavin, Thiamine, and optionally Inositol, Black Tea Extract and Nettle Extract. The various extracts are prepared according to traditional procedures, and then combined in a suitable formulation for administration to the patient for treatment of male, female and adolescent pattern hair loss.

The formulations of the present invention may be provided in topical lotion, cream, gel or tonic form, which will be suitable for administration to the patient. The amount of each of the individual components present in the formulation is selected for optimum therapeutic effect. The formulation contains the following ranges of active ingredients:
Green Tea Leaf Extract 75-320 mg
Epigallocatechin Gallate (EGCG) 15-85 mg
Polyphenols 30-85 mg
Vitamin E 50-65 mg
Folic Acid 0.72-0.85 mg
Zinc (as Oxide) 0.40-0.42 mg
Calcium Pantothenate 0.35-0.38 mg
Thiamine 0.25-0.28 mg
Copper 0.20-0.23 mg
Niacin 0.045-0.047 mg
Riboflavin 0.025-0.027 mg
Vitamin B12 0.0013-0.0017 mg
Biotin 0.0006-0.0008 mg
Inositol 30-100 mg
Black Tea Extract 27-100 mg
Nettle Extract 75-100 mg

Preferably, the formulation contains:
Green Tea Leaf Extract 220 mg
Epigallocatechin Gallate (EGCG) 25 mg
Polyphenols 40 mg
Vitamin E 55 mg
Folic Acid 0.78 mg
Zinc (as oxide) 0.44 mg
Calcium Pantothenate 0.40 mg
Thiamine 0.30 mg
Copper 0.21 mg
Niacin 0.047 mg
Riboflavin 0.028 mg
Vitamin B12 0.0010 mg
Biotin 0.0008 mg
Nettle Extract 50 mg-100 mg

The formulation for male facial skin treatment contains:
Green Tea Leaf Extract 105 mg
Epigallocatechin gallate (EGCG) 65 mg
Polyphenolslyphenols 45 mg
Vitamin E 60 mg
Folic Acid 0.75 mg
Calcium Pantothenate 0.36 mg
Thiamine 0.27 mg
Niacin 0.045 mg
Riboflavin 0.025 mg
Vitamin B12 0.0011 mg
Biotin 0.0007 mg

This dosage is ideally administered by applying one or two dosages per day each containing the preferred formulation as set out above.

The formulations of the present invention are thought to effect male, female and adolescent pattern hair loss through interaction with the cells of the skin in the scalp. It is thought that the formulations of the present invention will stimulate weak cells, prevent the premature death of cells, and stimulate skin tissue and hair follicles. Improving the health of the scalp can lead to a reversal of the hair loss process normally associated with male, female and adolescent pattern hair loss. The effects of the present invention may be immediately apparent. This hair loss reduction formulation must be taken regularly in order to obtain maximum benefit in a short time. Some people will see hair growth quickly, while other may take longer. A small percentage may not see any growth at all but will still stop losing hair prematurely.

The male facial skin treatment formula will help give a more youthful look to facial skin by reducing and repairing skin damage due to UV and environmental hazards such as smoke. Age effects such as wrinkles and discoloration are also reduced and repaired.

Although various preferred embodiments of the present invention have been described herein in detail, it will be appreciated by those skilled in the art that variations may be made thereto without departing from the spirit of the invention or the scope of the appended claims. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which this invention pertains.

1. A natural formulation comprising a combination of Green Tea leaf extract, Polyphenolslyphenols, Epigallocatechin Gallate (EGCG), Vitamin E, Folic Acid, Copper (as Amino Acid Chelate), B12, Zinc (as Oxide), Calcium Pantothenate, Niacin, Biotin, Riboflavin, Thiamine for treatment of male, female and adolescent pattern hair loss,

2. A formulation according to claim 1 wherein the formulation further comprises of one or more other ingredients selected from natural herbal extracts.

3. A formulation especially for male facial skin conditions, the formulation comprising a combination Green Tea leaf extract, Polyphenols, Epigallocatechin Gallate (EGCG), Vitamin E, Folic Acid, B12, Niacin, Biotin, Riboflavin and Thiamine.
